(19) United States
(12) Patent Application Publication
(10) Pub. No.: US 2021/0246409 A1
(43) Pub. Date: Aug. 12, 2021

(54) MULTIDIRECTIONAL SHEAR STRESS APPARATUS

(71) Applicant: Board of Supervisors of Louisiana State University and Agricultural and Mechanical College, Baton Rouge, LA (US)

(72) Inventors: Kevin J. MCCARTHY, Shreveport, LA (US); Deborah MCCARTHY, Shreveport, LA (US)

(73) Assignee: Board of Supervisors of Louisiana State University and Agricultural and Mechanical College, Baton Rouge, LA (US)

(21) Appl. No.: 17/210,394

(22) Filed: Mar. 23, 2021

Related U.S. Application Data
(60) Provisional application No. 62/970,797, filed on Feb. 6, 2020.

(51) Int. Cl. C12M 1/42; C12M 1/34

(52) U.S. Cl. CPC C12M 35/04; C12M 41/34; C12M 41/12; C12M 41/42

(57) ABSTRACT

The presently disclosed invention relates to methods of creating a laminar and shear stress environment for cells and Multidirectional Shear Stress Apparatuses comprising a baseplate, a dish attached to the baseplate, a motor attached to the baseplate, the motor turning a shaft, a cone functionally and rotationally attached to the shaft, a first plurality of wells defined in the baseplate to receive coverslips

### CROSS REFERENCE TO RELATED APPLICATIONS/PRIORITY

The present invention claims priority to U.S. Provisional Patent Application No. 62/970,797 filed Feb. 6, 2020, which is incorporated by reference into the present disclosure as if fully restated herein. Any conflict between the incorporated material and the specific teachings of this disclosure shall be resolved in favor of the latter. Likewise, any conflict between an art-understood definition of a word or phrase and a definition of the word or phrase as specifically taught in this disclosure shall be resolved in favor of the latter.

### STATEMENT REGARDING FEDERALLY SPONSORED RESEARCH OR DEVELOPMENT

This invention was made with government support under Grant No. 5R01DK111958 awarded by the National Institutes of Health. The government has certain rights in the invention.

### BACKGROUND

Atherosclerosis is a major cause of death and morbidity in the United States and the world. On challenge with creating therapeutics is recreating in vitro conditions that cells experience in vivo. One challenging environment to recreate with cell cultures is the turbulent fluid flow that is found in many locations in the arteries that also experience atherosclerosis. For the foregoing reasons, there is a pressing need for a device that can create turbulent fluid flow environment for cell cultures.

### SUMMARY

Wherefore, it is an object of the present invention to overcome the above-mentioned shortcomings and drawbacks associated with the current technology.

The presently disclosed invention relates to methods of creating a laminar and shear stress environment for cells and Multidirectional Shear Stress Apparatuses comprising a baseplate, a dish attached to the baseplate, a motor attached to the baseplate, the motor turning a shaft, a cone functionally and rotationally attached to the shaft, a first plurality of wells defined in the baseplate to receive coverslips. According to a further embodiment the Multidirectional Shear Stress Apparatus further comprises a locking square disposed on one of a bottom surface of the dish and an upper baseplate surface, and a mating locking recess defined in the other of the bottom surface of the dish and the upper baseplate surface. According to a further embodiment the shaft and a spindle of the cone are coaxial along a central axis. According to a further embodiment a centering pin extends from a lower cone surface and mates with a center notch in the dish. According to a further embodiment a lower cone surface has a radially interior and substantially planar central circular cone section and a radially exterior oblique cone section. According to a further embodiment the oblique cone section extends linearly radially outwardly at a constant first angle of inclination from the circular cone section to a cone radially exterior circumference. According to a further embodiment the Multidirectional Shear Stress Apparatus further comprises a gantry stationarily positioning the motor with respect to the cone. According to a further embodiment the Multidirectional Shear Stress Apparatus further comprises a gap between an inner vertical wall of the dish and a cone radially exterior circumference, wherein the gap is between 0.50 mm and 3.00 mm. According to a further embodiment the Multidirectional Shear Stress Apparatus further comprises a coupler connecting the shaft to a spindle of the cone. According to a further embodiment the first plurality of wells is equidistant from a radial center of the dish, forming a first ring According to a further embodiment the Multidirectional Shear Stress Apparatus further comprises a second plurality of wells equidistant from the radial center of the dish and radially spaced from the first ring. According to a further embodiment the motor is a stepper motor. According to a further embodiment the Multidirectional Shear Stress Apparatus further comprises a sensor in the dish which measures one of flow speed, flow direction, flow speed and flow direction, temperature, and CO2 level. According to a further embodiment the Multidirectional Shear Stress Apparatus further comprises an upper dish surface having a radially interior and substantially planar central circular dish section and a radially exterior oblique dish section, with the first plurality of wells being disposed in the oblique dish section. According to a further embodiment a lower cone surface has an oblique cone section extending linearly radially outwardly at a constant first angle of inclination from a central circular cone section, and the oblique dish section extends linearly radially outwardly at a constant second angle of inclination from the central circular dish section, and the first angle of inclination is substantially the same as the second angle of inclination. According to a further embodiment the Multidirectional Shear Stress Apparatus further comprises a transparent well base in each of the first plurality of wells, allowing coverslips placed on the transparent well base to be viewed from below the Multidirectional Shear Stress Apparatus during operation of the Multidirectional Shear Stress Apparatus. According to a further embodiment the Multidirectional Shear Stress Apparatus further comprises a dish indentation defining a through hole in the baseplate sized to receive and outer vertical wall of the dish therewithin. According to a further embodiment the Multidirectional Shear Stress Apparatus further comprises support columns disposed at a plurality of locations around a perimeter of the dish indentation, the support columns defining a joist passage at each location for dish joists to fit at least partially within. According to a further embodiment dish joists extend from the outer vertical wall of the dish at a plurality of locations.

The presently disclosed invention further relates to methods generating laminar and sheer stress and Multidirectional Shear Stress Apparatuses comprising a baseplate, a dish attached to the baseplate, a stepper motor attached to the baseplate, the motor turning a shaft, a cone functionally and rotationally attached to the shaft, a first plurality of wells defined in the baseplate to receive coverslips, a dish indentation sized to receive a vertical outer wall of the dish, one of (a) a locking square disposed on one of a bottom surface of the dish and an upper baseplate surface, and a mating locking recess defined in the other of the bottom surface of the dish and the upper baseplate surface, and (b) the dish indentation defining a through hole in the baseplate sized to receive and outer vertical wall of the dish therewithin and support columns disposed at a plurality of locations around a perimeter of the dish indentation, the support columns defining a joist passage at each location for dish joists to fit at least partially within, a spindle extending from a radial center of the cone, the spindle being coaxial with the shaft along a central axis, a coupler connecting the shaft to a spindle of the cone, a cross brace encircling and bracing the spindle, a gantry stationarily positioning the motor with respect to the cone, a gap between an inner vertical wall of the dish and a cone radially exterior circumference, wherein the gap is between 0.50 mm and 3.00 mm, a lower cone surface having a radially interior and substantially planar central circular cone section and a radially exterior oblique cone section, the oblique cone section extends linearly radially outwardly at a constant first angle of inclination from the circular cone section to a cone radially exterior circumference, a centering pin extending from the lower cone surface and mating with a center notch in the dish, the first plurality of wells being equidistant from a radial center of the dish and forming a first ring, a second plurality of wells equidistant from the radial center of the dish and radially spaced from the first ring, a sensor in the dish which measures one of flow speed, flow direction, flow speed and flow direction, temperature, and CO2 level, a well base in each of the wells being one of opaque, translucent, and transparent, where a transparent well base allows cells on coverslips placed on the transparent well base to be viewed from below the Multidirectional Shear Stress Apparatus during operation of the Multidirectional Shear Stress Apparatus, and a cover being coaxial with the cross brace and extending radially substantially past an internal diameter of the inner vertical wall of the dish.

Various objects, features, aspects, and advantages of the present invention will become more apparent from the following detailed description of preferred embodiments of the invention, along with the accompanying drawings in which like numerals represent like components. The present invention may address one or more of the problems and deficiencies of the current technology discussed above. However, it is contemplated that the invention may prove useful in addressing other problems and deficiencies in a number of technical areas. Therefore, the claimed invention should not necessarily be construed as limited to addressing any of the particular problems or deficiencies discussed herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings, which are incorporated in and constitute a part of the specification, illustrate various embodiments of the invention and together with the general description of the invention given above and the detailed description of the drawings given below, serve to explain the principles of the invention. It is to be appreciated that the accompanying drawings are not necessarily to scale since the emphasis is instead placed on illustrating the principles of the invention. The invention will now be described, by way of example, with reference to the accompanying drawings in which:

FIG. 1 is a photograph of a first full sized embodiment of the Multidirectional Shear Stress Apparatus, according to the presently disclosed invention;

FIG. 2 is a photograph of a second micro embodiment of the Multidirectional Shear Stress Apparatus loaded onto a microscope stage , according to the presently disclosed invention;

FIG. 3 is a photograph of the Multidirectional Shear Stress Apparatus of FIG. 2, showing the a more substantial portion of the microscope;

FIG. 4 is a schematic isomeric view of the Multidirectional Shear Stress Apparatus of FIG. 1;

FIGS. 5A and 5B are respectively a schematic side view and a schematic front plan view of the Multidirectional Shear Stress Apparatus of FIG. 1;

FIG. 6 is a schematic top plan view of the Multidirectional Shear Stress Apparatus of FIG. 1;

FIG. 7 is a schematic top front perspective view of the dish of the Multidirectional Shear Stress Apparatus of FIG. 1;

FIGS. 8A and 8B are respectively a sectional view and a bottom front perspective view of the dish of FIG. 7

FIG. 9 is a schematic bottom perspective view of the of the Multidirectional Shear Stress Apparatus of FIG. 1;

FIG. 10 is a sectional view of the baseplate, support, and gantry of the Multidirectional Shear Stress Apparatus of FIG. 1;

FIG. 11 is a schematic top front perspective view of the baseplate, support, and gantry of FIG. 10

FIGS. 12A and 12B are respectively a schematic top front perspective view of the motor mount of the Multidirectional Shear Stress Apparatus of FIG. 1, and a schematic top side perspective view of the Multidirectional Shear Stress Apparatus of FIG. 1;

FIGS. 13A and 13B are respectively a schematic bottom front perspective view and a sectional view of the coupler of the Multidirectional Shear Stress Apparatus of FIG. 1;

FIGS. 14A and 14B are respectively a schematic front top perspective view and a sectional view of cone of the Multidirectional Shear Stress Apparatus of FIG. 1;

FIG. 15 is a schematic isomeric view of the Multidirectional Shear Stress Apparatus of FIG. 2;

FIG. 16 is a sectional view of the Multidirectional Shear Stress Apparatus of FIG. 15;

FIG. 17 is a schematic perspective view of the dish view of the Multidirectional Shear Stress Apparatus of FIG. 2;

FIGS. 18A and 18B are respectively a schematic top front perspective view of the cover and a schematic top front perspective view of the cone of the Multidirectional Shear Stress Apparatus of FIG. 2;

FIG. 19 is a schematic perspective view of the cross brace of the Multidirectional Shear Stress Apparatus of FIGS. 1 and 2;

FIG. 20 is a schematic top front perspective view of the baseplate of the Multidirectional Shear Stress Apparatus of FIG. 2;

FIG. 21 is a sectional view of the of the Multidirectional Shear Stress Apparatus of FIG. 6, along sectional line F21

FIG. 22 is a sectional view of the of the Multidirectional Shear Stress Apparatus of FIG. 6, along sectional line F22

FIG. 23 is a schematic partial sectional view of a further embodiment of the dish and cone of the section view of the of the Multidirectional Shear Stress Apparatuses of FIGS. 1 and 2;

FIGS. 24A-24F are photomicrographs of two different type of glomerular podocyte cells subjected to three different environments in the Multidirectional Shear Stress Apparatus of FIG. 1, where FIG. 24A is HS+ stat, FIG. 24B is HS− stat, FIG. 24C is HS+ lam; FIG. 24D is HS− lam, FIG. 24E is HS+ turb, and FIG. 24F is HS− turb, where HS+ is wild type cell, HS− is mutant cell, stat is static medium, lam is laminar flow medium, and turb is turbulent flow medium; and

FIG. 25 is a photograph of the Multidirectional Shear Stress Apparatus of FIG. 1 and the Multidirectional Shear Stress Apparatus of FIG. 2 in the same image for comparison.

### DETAILED DESCRIPTION

The present invention will be understood by reference to the following detailed description, which should be read in conjunction with the appended drawings. It is to be appreciated that the following detailed description of various embodiments is by way of example only and is not meant to limit, in any way, the scope of the present invention. In the summary above, in the following detailed description, in the claims below, and in the accompanying drawings, reference is made to particular features (including method steps) of the present invention. It is to be understood that the disclosure of the invention in this specification includes all possible combinations of such particular features, not just those explicitly described. For example, where a particular feature is disclosed in the context of a particular aspect or embodiment of the invention or a particular claim, that feature can also be used, to the extent possible, in combination with and/or in the context of other particular aspects and embodiments of the invention, and in the invention generally. The term “comprises” and grammatical equivalents thereof are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

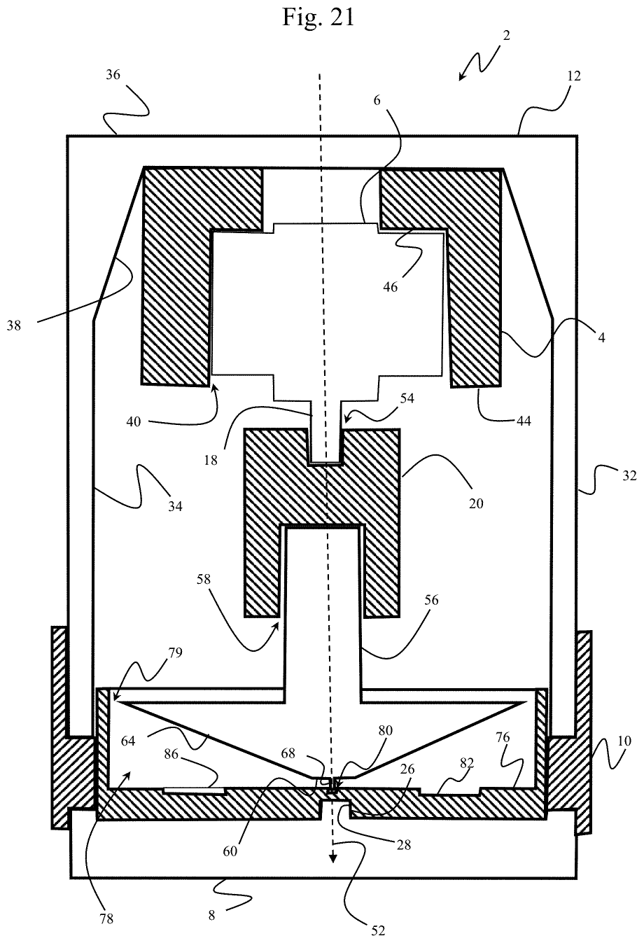

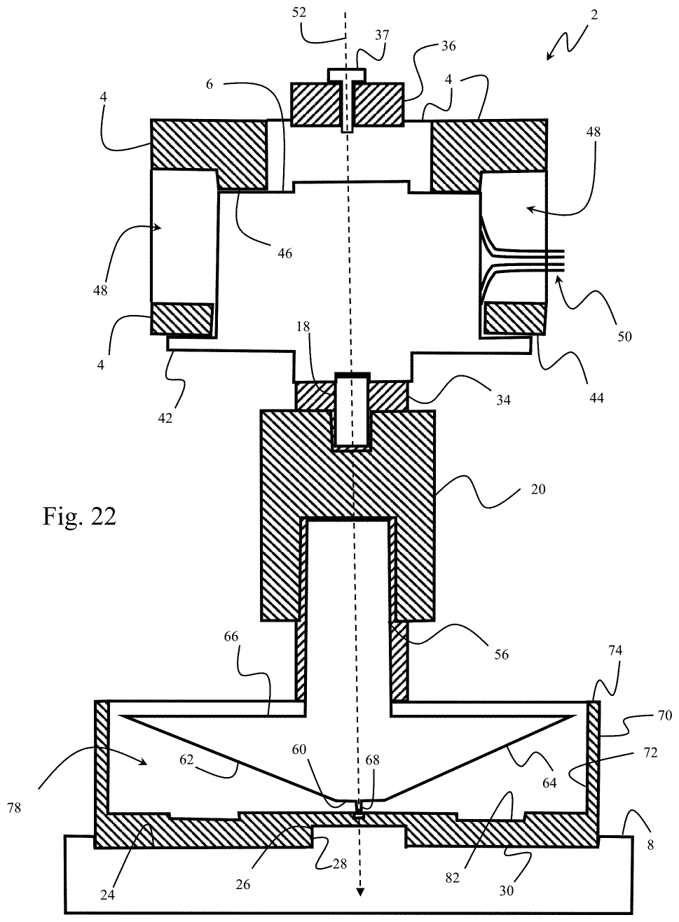

The term “at least” followed by a number is used herein to denote the start of a range beginning with that number (which may be a range having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40% means 40% or less than 40%. When, in this specification, a range is given as “(a first number) to (a second number)” or “(a first number)-(a second number),” this means a range whose lower limit is the first number and whose upper limit is the second number. For example, 25 to 100 mm means a range whose lower limit is 25 mm, and whose upper limit is 100 mm. Where spatial directions are given, for example above, below, top, and bottom, such directions refer to the Multidirectional Shear Stress Apparatus 2 as represented on FIGS. 21 and 22, unless identified otherwise.

The embodiments set forth the below represent the necessary information to enable those skilled in the art to practice the invention and illustrate the best mode of practicing the invention. For the measurements listed, embodiments including measurements plus or minus the measurement times 5%, 10%, 20%, 50% and 75% are also contemplated. For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

In addition, the invention does not require that all the advantageous features and all the advantages of any of the embodiments need to be incorporated into every embodiment of the invention.

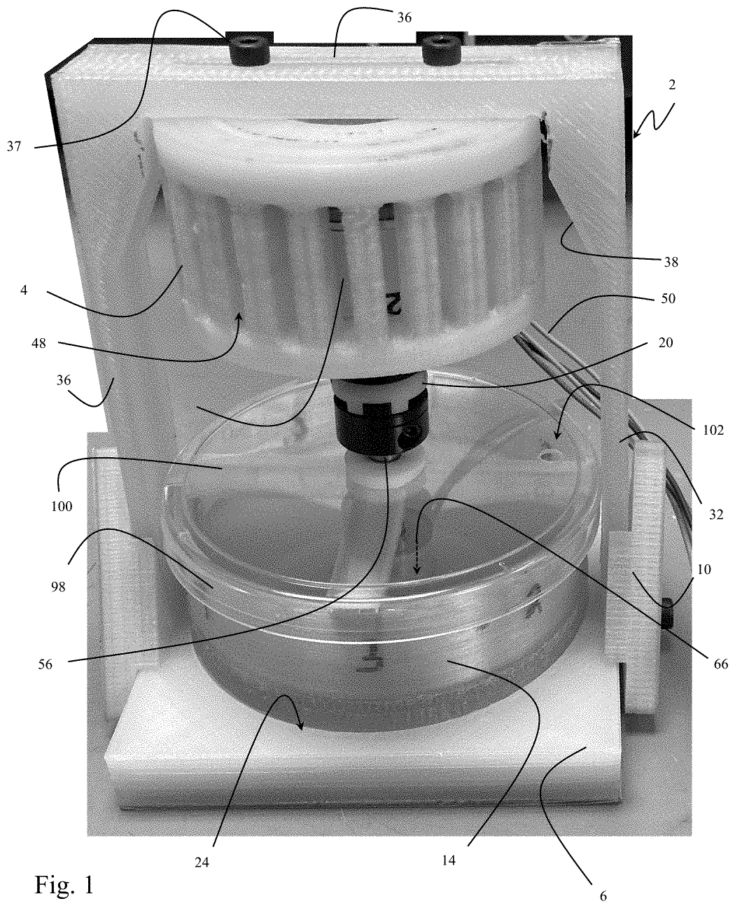

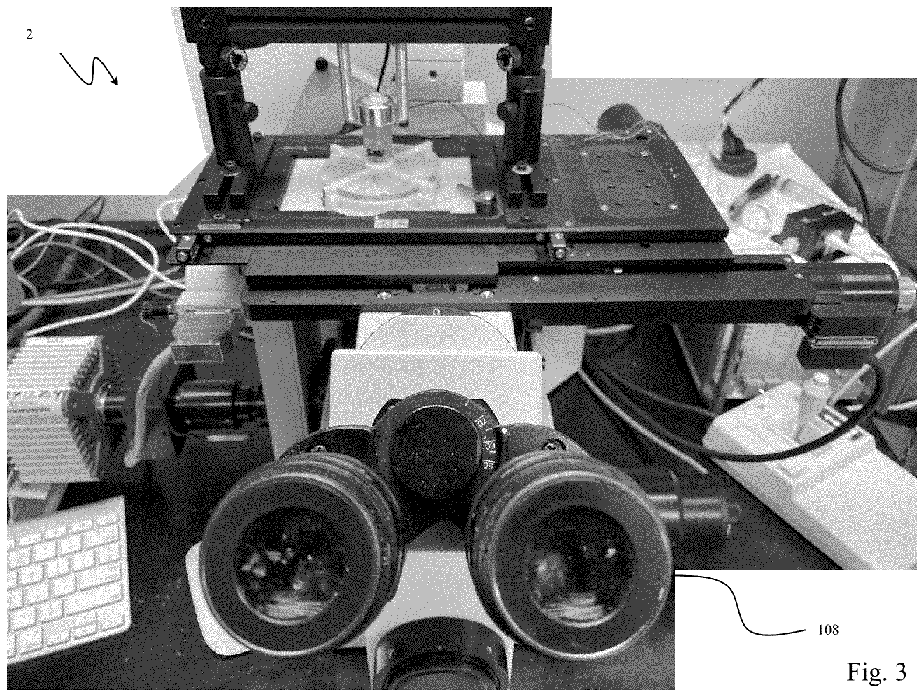

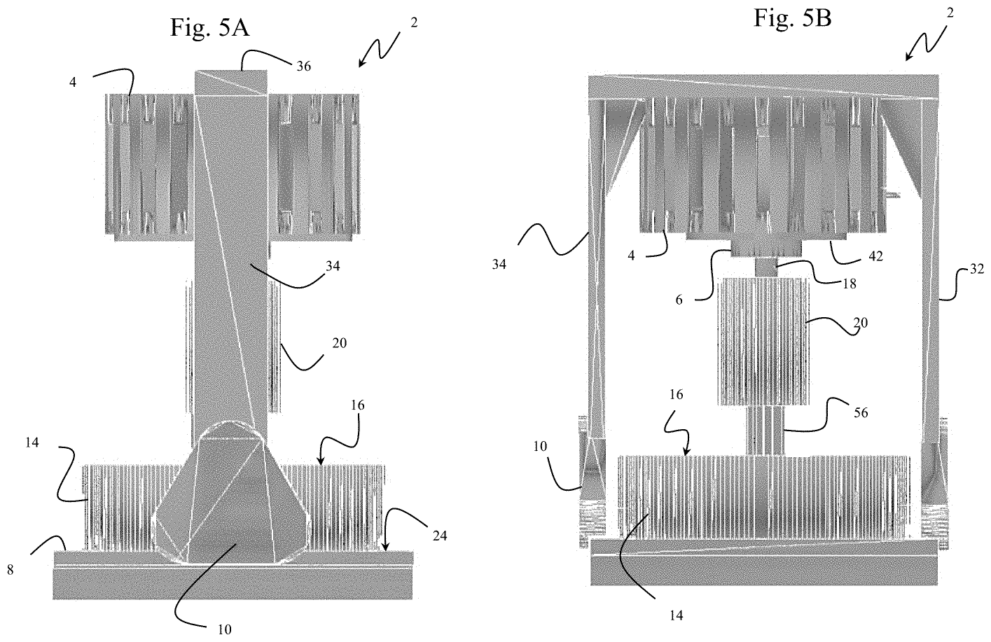

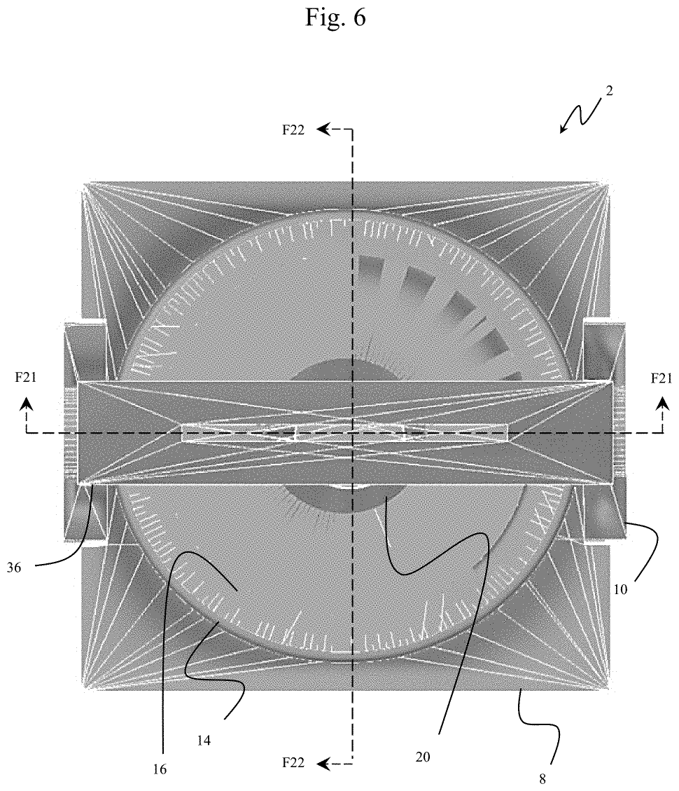

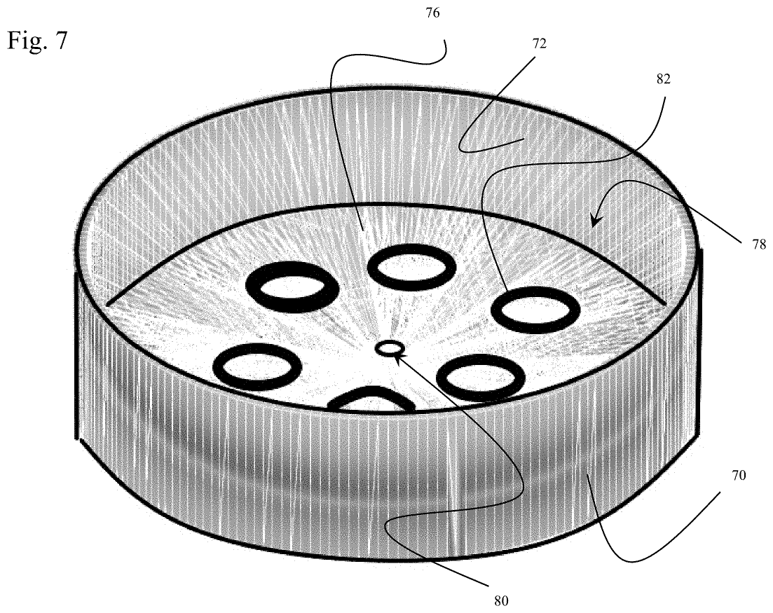

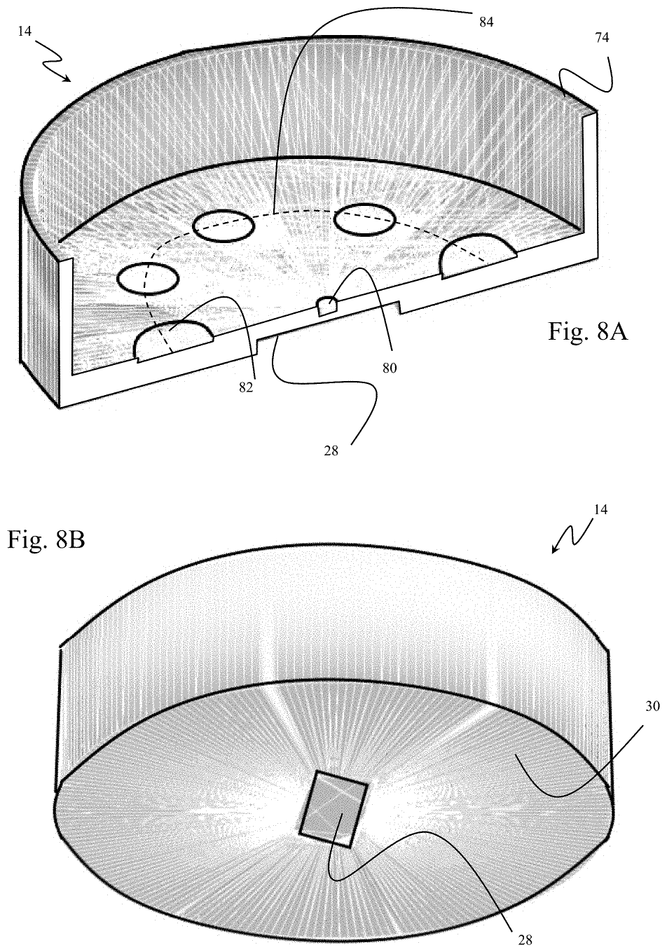

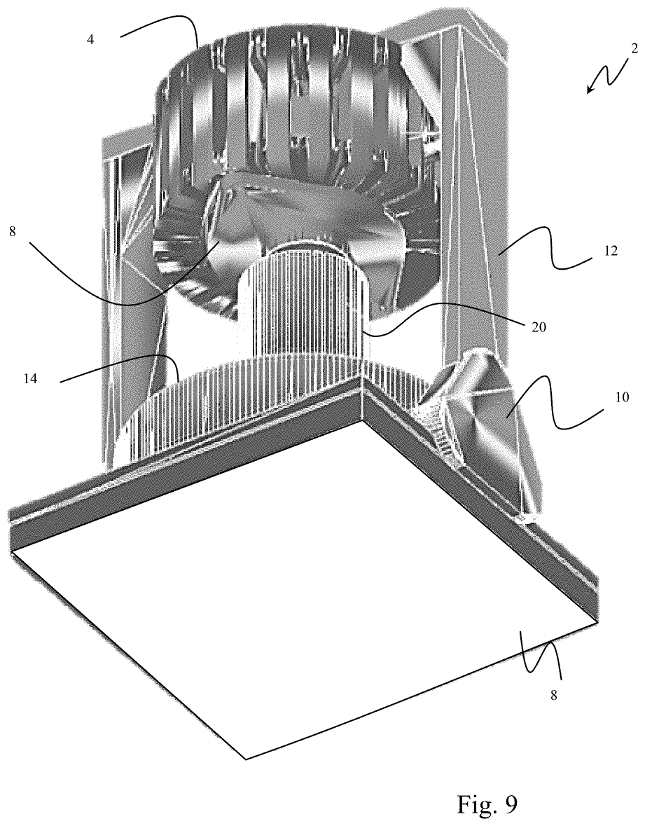

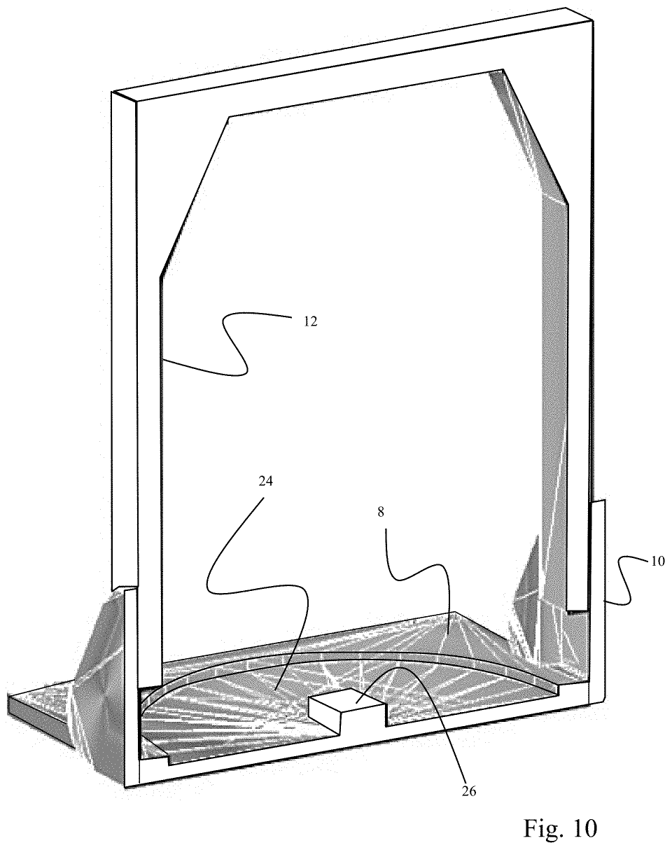

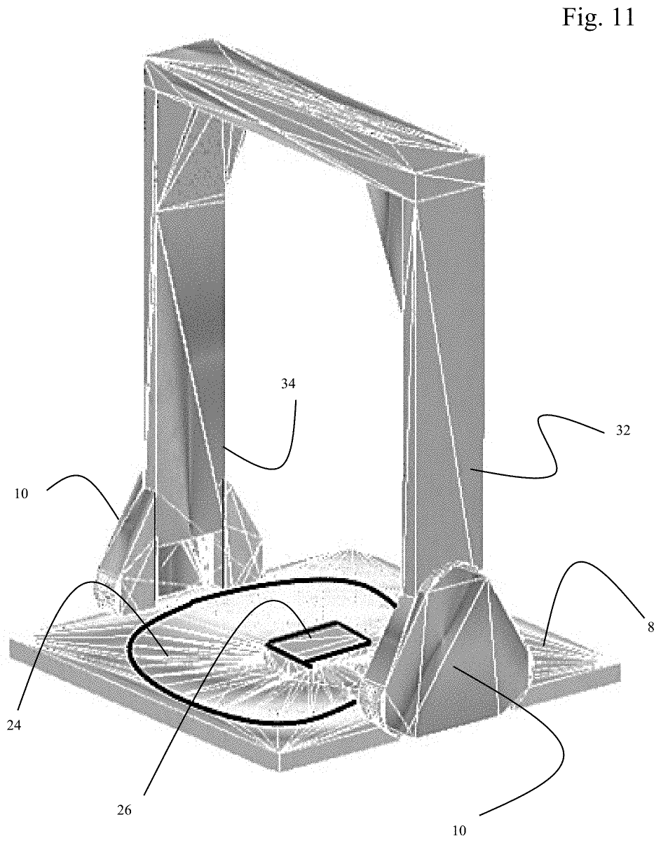

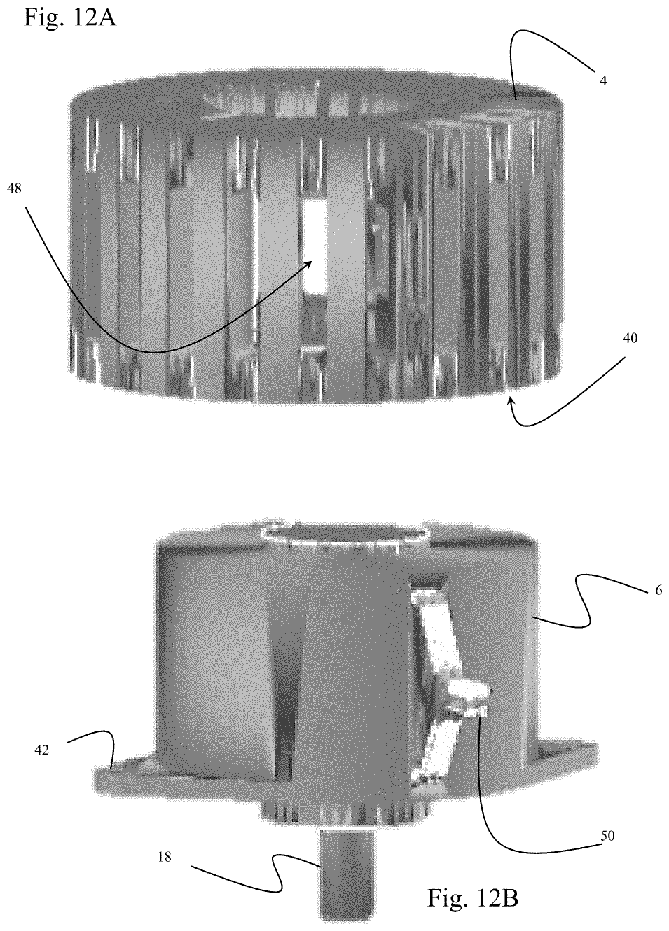

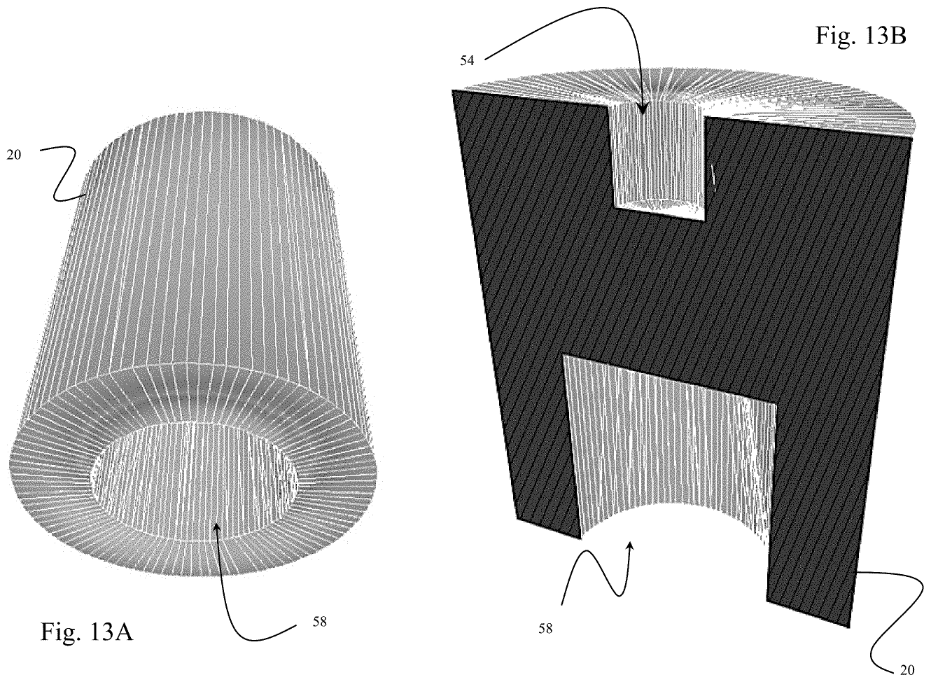

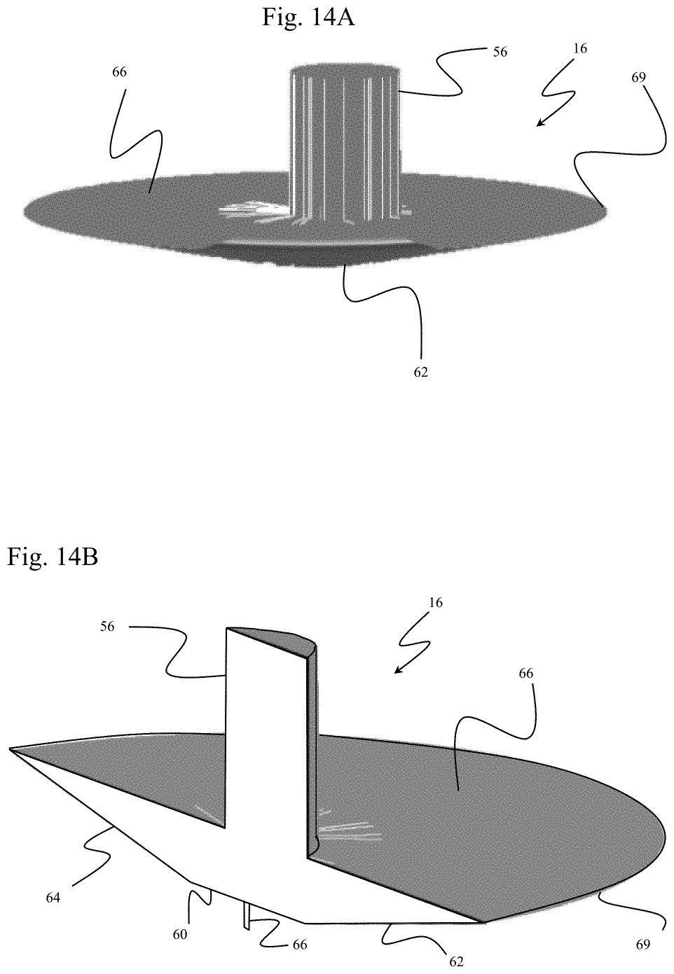

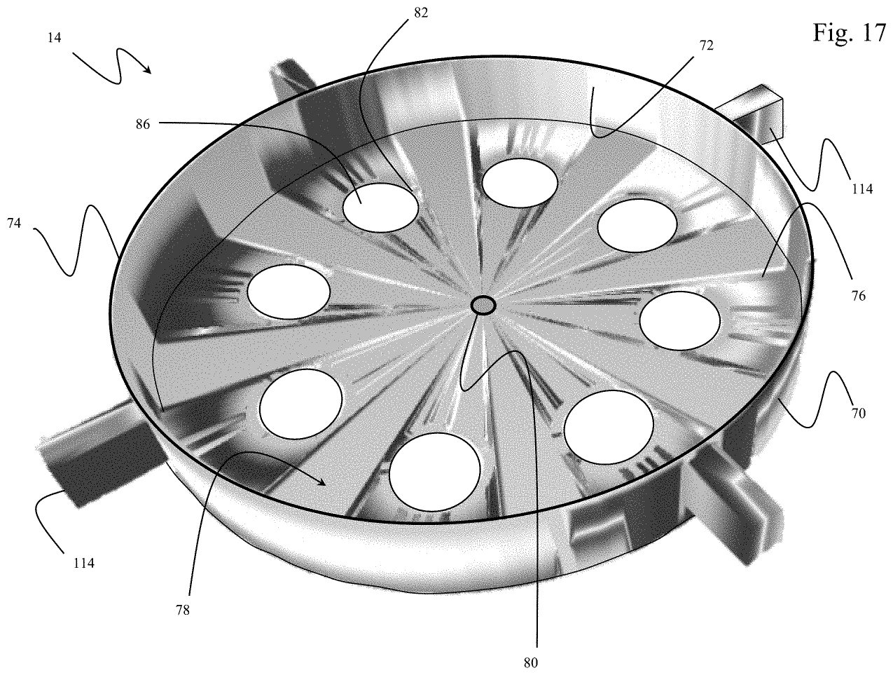

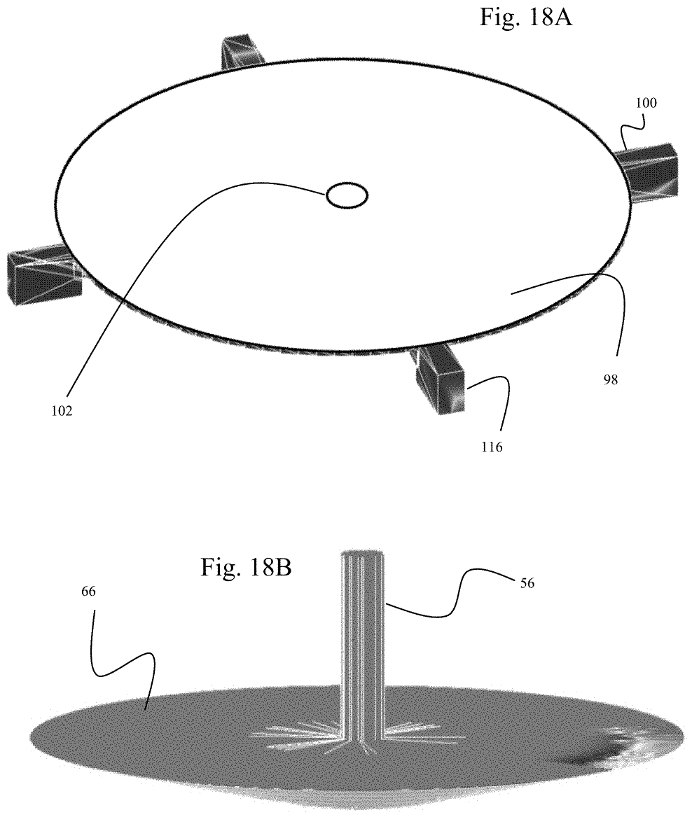

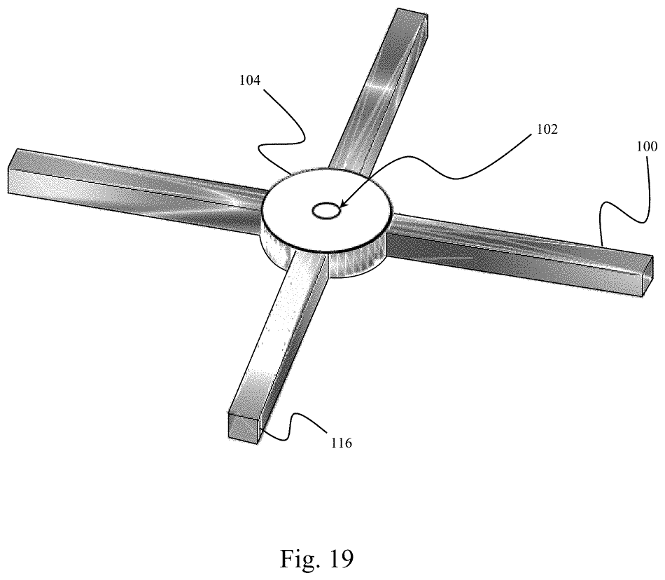

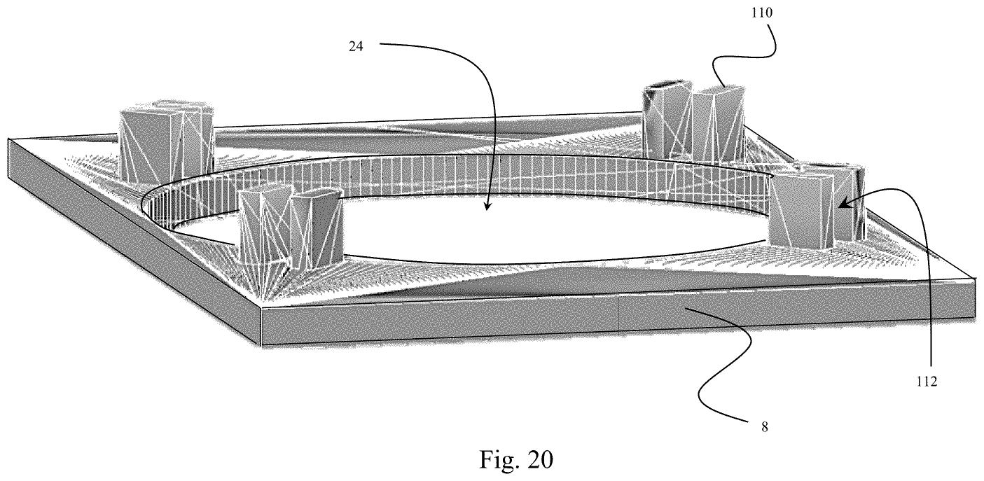

Turning now to the FIGS. 1-25, a brief description concerning the various components of the various embodiments of the presently disclosed invention will now be briefly discussed. As can be seen in FIGS. 1 and 21-23, for example, the first embodiment of the multidirectional shear stress apparatus 2 includes a motor mount 4, a stepper motor 6, a baseplate 8 and support 10, a gantry 12, a dish 14, a cone 16, and a shaft 18 and coupler 20.

The baseplate 8 is a substantially planar surface, preferably wide enough to provide the Multidirectional Shear Stress Apparatus 2 with a stability as the Multidirectional Shear Stress Apparatus 2 is functioning. An upper surface 22 of the baseplate 8 has a dish indentation 24, recessed approximately 10-50 mm below the upper surface 22 of the baseplate 8. In the center of the dish indentation 24, a locking square 26 extends upward from the surface of the dish indentation 24. The locking square 26 fits within a locking recess 28 defined in a bottom surface 30 of the dish 14. When the dish 14 is placed on the baseplate 8, in the dish indentation 24, the locking square 26 mates with the locking recess 26, and the dish 14 is prevented from rotating with respect to the baseplate 8. This adds functionality when the cone 16 rotates with respect to the dish 14 upon activation of the Multidirectional Shear Stress Apparatus 2. It is contemplated that a single locking square 26 may be shaped alternatively as not having a square horizontal cross section, but as another non-circular shaped locking square 26 (such as a triangular horizontal cross section) that mates with a correspondingly non-circularly shaped locking recess 28 (such as a triangular horizontal cross section). Additionally, if multiple locking squares 26 are provided, they may be relatively any shape, including circular, and mate with likewise shaped locking recesses 28 and prevent relative rotation of the dish 14 and the baseplate 8. In the embodiment shown the baseplate is 9.9 cm wide, 9.9 cm long, and 0.767 cm high.

The baseplate 8 includes a pair of supports 10 that support and engage with the gantry 12. The supports 10 in the embodiment shown are substantially triangular in shape to provide strength in supporting the gantry 12, while minimizing the material required.

Gantry 12 is a frame that spaces the motor mount 4 above the cone 16, and holds the motor mount 4 steady while the motor 6 is operating. The gantry 12 has first and second legs 32, 34, lower portions of each attached to the baseplate 8 adjacent to the supports 10. The first and second legs 32, 34 are spaced wider than the width of the dish 14. Upper portions of each leg are attached to a crossbeam 36, upon which the motor mount 4 is attached with screws, nuts and bolts, or other fasteners 37. Brackets 38 are preferably located at the attachment of the legs 32, 34 and crossbeam 36 to increase structural integrity of the gantry 12. The crossbeam 36 is positioned high enough to allow the motor mount 4, motor 6, shaft 18, coupler 20, cone 16, and the dish 14 to fit there below. In the embodiment shown, the first and second arms 32 ,34 are 2.0 cm wide×0.5 cm thick/long×11.5 cm tall, and the crossbeam 36 is 2.0 cm wide×10 cm long×0.75 cm tall.

The motor mount 4 is substantially torus shaped. The motor mount 4 has an interior motor recess 40 that receives the motor, a motor coupling 42 along a lower shoulder ring 44 that the motor is coupled to, and an upper shoulder ring 46, which extends radially inwardly further than the lower shoulder ring 44, that the motor 6 abuts against when the motor 6 is attached to the motor coupling 42. The motor mount 6 preferably has a plurality of through longitudinal slits 48 defined in the circumference of the motor mount 6, which allow air flow from outside of the motor mount 6 to directly contact the motor 6, and thus heat dissipation from the motor 6 while the motor 6 is operating, while still ensuring structural integrity of the motor mount 6.

The motor 6 is a stepper motor 6. It fits in the interior motor recess 40 of the motor mount 6, and has electrical power and control wires 50 extending out from the motor 6, and a shaft 18 extending downward from the motor.

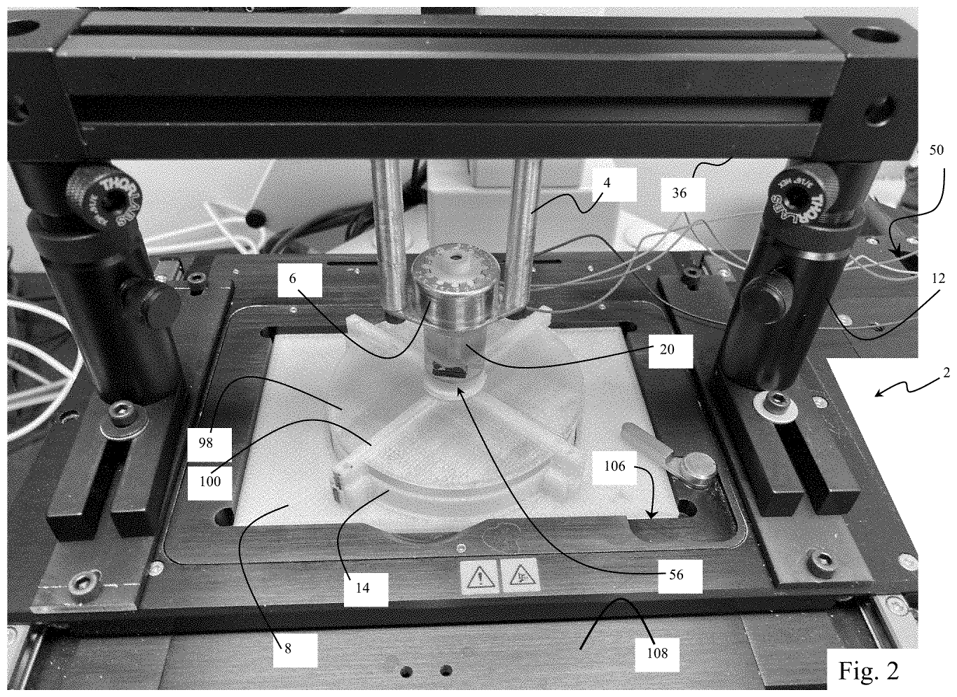

The motors 8 shown in FIGS. 1 and 2 are bipolar stepper motors 8, the preferred motor 6 for the Multidirectional Shear Stress Apparatus 2. The motor 6 shown in FIG. 1 has a step angle of 7.5 degrees, a holding torque of 11.3 Ncm, a detent torque of 0.64 Ncm, a rotor inertia of 25 gcm², and a chassis diameter of 46 mm. Other stepper motors are 10-watt, 15-degree step angles.

An advantage to the motor 6 being a stepper motor for moving the cone 16 is that it allows for implementation of bilateral movement protocols whose degree, rate, and duration of motion ramping (e.g., Acceleration, deceleration, and pausing) are all operator-controlled variables that can be controlled via programming, and are capable of software-controlled movement in a repeatable, precise, bidirectional manner. Attempting to used other motors 8 in the same way as the stepper motor 6 may not be possible, or may lead to quick burnout out the motors. The Multidirectional Shear Stress Apparatus 2 with the stepper motor 6 is capable of forward and reverse motions of the cone 16 at multiple speeds, accel-decel rates, and pause intervals as dictated by the needs of the studies. Further, the Multidirectional Shear Stress Apparatus 2 is capable of running in a manner that is capable of generating both laminar and turbulent shear without changing the basic configuration of the device.

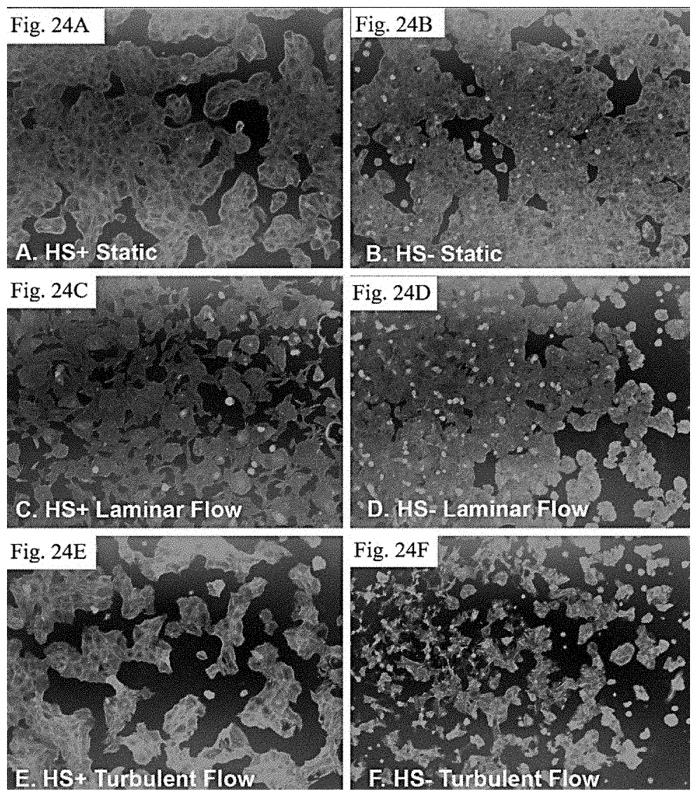

For the inventors' initial studies on comparing the effects of laminar or shear (turbulent) flow on the behavior of cells (podocytes), with results shown in FIGS. 24A-24F the protocol the inventors used for laminar flow was the cone was programmed to rotate at 120 RPM for 6 hours in one direction. The protocol the inventors used to subject cells to turbulent flow, was the cone was programmed to rotate at 120 RPM but only permitted to rotate 6.25 rotations until evoking a change in direction. That “back and forth” motion was maintained for 6 hours.

The shaft 18 extends downward from the motor 6, terminating in and mating with the coupler 20. The shaft 18 defines a central axis 52. The shaft 18 transmits rotation speed, direction, and torque from the motor 6 to the coupler 20. The coupler receives a terminal portion of the shaft 18 in an upper coupler concavity 54, and receives an upper portion of a spindle 56 portion of the cone 16 into a lower coupler concavity 58. The spindle 56 is coaxial with the central axis 52. The cone 16 is attached at an upper spindle 56 portion to the coupler 20 in the lower coupler concavity 58. The coupler 20 functionally connects the shaft 18 to the spindle 56, transmitting rotation speed, direction, and torque from the shaft 18, through the coupler 20 to the spindle 56 of the cone 16. The design of the coupler 20 is such that it allows for minor degrees of alignment offset between the shaft 18 and the spindle 56. This can be accomplished, for example, by mechanical coupling (e.g. Oldham-style couplings) or via magnetic couplers interfaced between the shaft 18 and the spindle 56, for example.

The cone 16 is substantially shaped as a broad cone with a flat central circular cone section 60 along the lower cone surface 62, an oblique cone section 64 along the lower cone surface 62, a planar circular upper cone surface 66, and a cylindrical spindle 56 section extending upwards from the center of the circular upper cone surface 66. At a central location of the central circular cone section 60, a centering pin 68 extends downward and is coaxial with the central axis 52 when the Multidirectional Shear Stress Apparatus 2 is operating. The centering pin 68 is preferably stainless steel and extends from 0.5 to 1.0 mm from the central circular cone section 60. The oblique cone section 64 extends linearly at a first angle of inclination a above the horizontal. The angle a is preferably between 1% and 10%, more preferably between 2% and 6% and most preferably 4%. The lower cone surface 62 is preferably polished smooth to decrease unintended turbulence, having a Ra of preferably between 1.0 and 0.01 μm, more preferably between 0.2 and 0.05 μm, and most preferably 0.1 μm.

In the embodiments shown in FIGS. 1 and 2, the lower cone surface 62 is smooth to permit the exploration of one fluidic component (flow) in a single experiment. To add additional parameters to an experiment, the lower cone surface 62 can be altered to evoke a pressure wave concurrently with cone 16 rotation to replicate the pressure wave/flow pattern seen by cells lining the larger systemic arterial tree. In this case, the lower cone surface 62 can be made to define one or more waves, such that moving along an inscribed circle at a given radial distance from the center of the cone, the lower cone surface 62 would become closer in proximity to the upper dish surface 76 and then farther from the upper dish surface 76 for each wave, inducing a local, transient pressure wave. The waves may be manifest as one or more convex radial protrusions protruding downward from the oblique cone section 64, and/or one or more concave radial indentations indented in or defined in the oblique cone section 64. The radial protrusions or radial indentions extending linearly from an internal radius to an external radius. The waves may be just concave, just convex, or include both concave and convex portions. If the waves are both concave and convex, then preferably the convex portion is directly adjacent to the concave portion, and the convex wave portion preferably transitions seamlessly into the concave portion. The waves preferably stretch substantially across an entire radial length of the oblique cone section 64, from the radially exterior circumference of the cone 69 inwardly to the radially outer edge of the central circular section 60, and a width of preferably between 1 degree and 30 degrees of a cone circumference at any point along the radial length of the oblique cone section 64. In one embodiment of this waved cone, a graph of an inscribed circle along the oblique cone section 64 at a given radial distance from the center of the cone 16, with distance from the upper dish surface 76 as the y axis and a distance along the circumference of the inscribed circle as the x axis, forming a substantially sinusoidal wave.

In the embodiment of the cone shown in FIG. 1, the upper cone surface preferably has a radius of 4.20 cm, and a radius of the central circular cone section of 1.0 cm, and a height of 0.70 cm.

The dish 14 is substantially bowl shaped, and has the bottom dish surface 22, an outer vertical wall 70, an inner vertical wall 72, a rim 74 connecting the upper portions of the outer vertical wall 70 and inner vertical wall 72, and an upper dish surface 76. A space partially enclosed and defined by the inner vertical wall 72 and the upper dish surface is the interior 78 of the dish 14. A space between the inner vertical wall 72 and the cone radially exterior circumference 69 defines a gap 79. In a central location in upper dish surface 76, coaxial with the central axis, a center notch 80 is defined. The center notch 80 is shaped to receive the centering pin 68 of the cone 16, and maintain the cone 16 coaxial with the central axis 52 as the Multidirectional Shear Stress Apparatus 2 operates.

One or more wells 82 are defined in the upper dish surface 76. In the first embodiment, eight wells 82 are defined in the upper dish surface 76 to define a first ring 84. The wells 82 are bores into the upper dish surface 76 of a radius and depth to fit a preferably circular shaped coverslip 86, such that when the coverslip 86 is placed into the well 82, an upper surface of the coverslip 86 is coplanar with the upper dish surface 76. In further embodiments, there may be additional rings of wells, for example, second and third rings 88, 90. The wells 82 in each ring 84, 88, 90 are equidistant from the radial center of the upper dish surface 76. Each ring 84, 88, 90 is at least partially and preferably wholly radially spaced from each other ring 84, 88, 90. In a first embodiment, the upper dish surface 76 is circular and substantially planar. The upper dish surface 76 and part or all of inner vertical wall 72 is preferably polished smooth to decrease unintended turbulence, having a Ra of preferably between 1.0 and 0.01 μm, more preferably between 0.2 and 0.05 μm, and most preferably 0.1 μm.

In the embodiment shown in FIG. 1, the dish 14 measures 4.30 cm inside diameter, 4.40 cm outside diameter, and 2.50 cm height. The dish 14 bottom has a 0.50 cm thickness between the dish upper surface 76 and the dish lower surface 30. The dish 14 has 8 wells 82 that accept 10 mm circular optical glass coverslips 86. The wells 82 have an individual well radius of 0.62 cm, a depth of 0.10 cm, and a distance from center of plate of 2.5 cm.

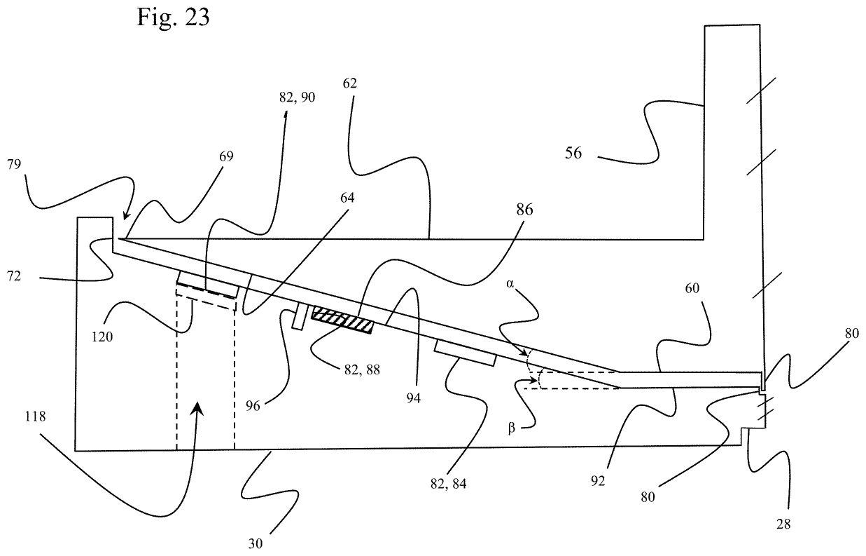

In a further embodiment, the upper dish surface 76 partially mirrors the shape of lower cone surface 62, and has a flat central circular dish section 92 along the upper dish surface 76, with the radial center of the center notch 80 being the radial center of the upper dish surface 76. An oblique dish section 94 of the upper dish surface 76 extends outward and upwards from the central circular dish section 92. The oblique dish section 94 extends linearly at a second angle of inclination β above the horizontal. The second angle of inclination β is preferably between 1% and 10%, more preferably between 2% and 6% and most preferably 4%, and preferably is the same angle as the first angle of inclination α. In the embodiments where the upper dish surface 76 conforms with the first angle of inclination α of the cone 16 after being radially past the central circular dish section 92, this provides an equidistant space between the lower cone surface 66 and the upper dish surface 76, from the center of the device to its periphery. By doing so, the entire fluidics of the medium under flow would be constrained to a single dimensional component, thus minimizing variables and allowing for greater certainty in and reproducibility of experiments. This minimization of variables is especially beneficial when there are multiple rings 84, 88, 90 of wells 82, as shown in FIG. 23.

In further embodiments there are sensors 96 in place in the dish 14, preferably adjacent to but coplanar with or beneath the upper dish surface 76, that may measure medium velocity, medium temperature, CO2 level, O2 level, and pH, for example. In further embodiments, there may be multiple sensors 96 along a radial path that tracks, for example, the flow speed and direction, and other variables, at various radial locations in the dish 14.

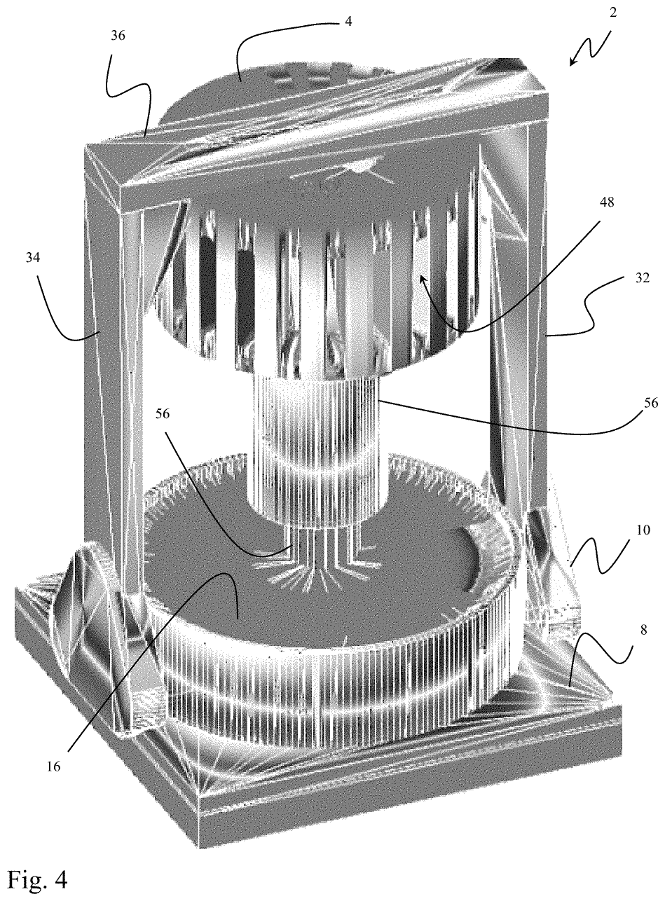

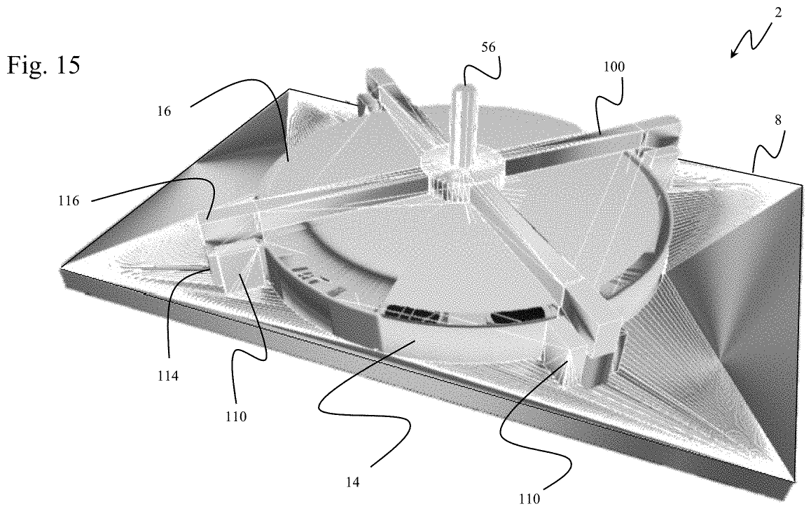

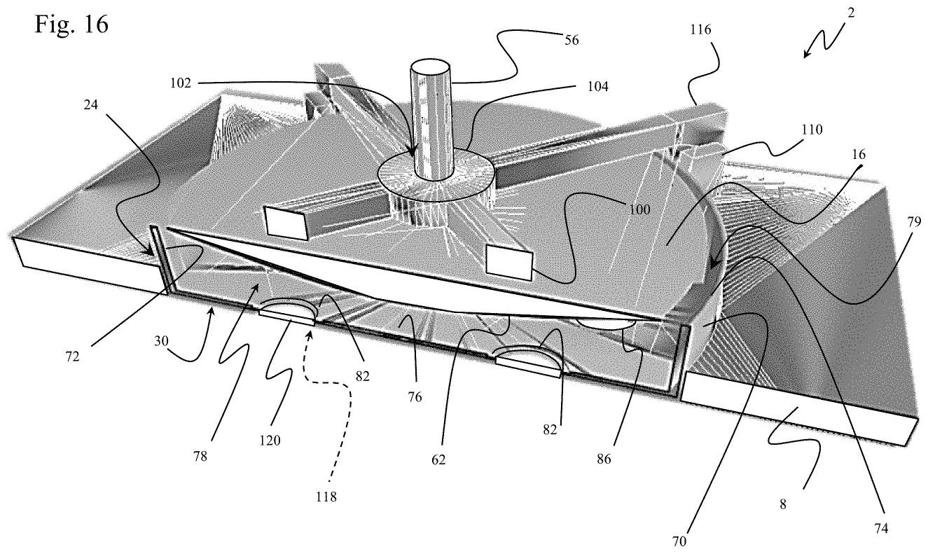

Testing of the prototype in standard cell culture incubators has shown that the medium is subject to some evaporative loss during operation. To minimize/eliminate evaporative loss a cover was designed (omitted from FIGS. 4-6, 9, 21, and 22, and partially omitted from FIGS. 15 and 16 for clarity) The cover 98 is preferably integrated into a cross brace 100 to cap the cell plate, and an access hole to allow access into the interior of the dish 78 during operation without having to remove the cover 98. The cover 98 preferably has a circumference larger than an inside diameter of the dish 14, so that the cap may rest on the rim of the dish and provide at least a partial barrier. In the embodiment of FIG. 1, the cover 98 is shaped as a horizontal circular cap with a lip that extends downward around a circumference of the cover 98, mating with the top of the outer vertical wall 70 of the dish 14. The cross beams of the cross brace 100 are attached to the underside of the cover 98, and are sized to have a clearance fit, preferably a sliding clearance fit with the inner vertical walls 72 of the dish 14. In the embodiment of FIG. 2, the cover may forgo the lip, and the cross brace 100 may be above, below, or, as in FIG. 2, partially integrated with and intersecting the cover 98. A central through hole in the cover 98 defines a spindle passage 102. The cross brace 100 preferably has a center core 104, from which the legs of the cross brace 100 extend. The center of center core 104, through which the spindle 56 passes, also defines the spindle passage 102. The center core 104 gives the cross brace 100 and cover 98 increased structural integrity and gives the cone 16 increased stability when the Multidirectional Shear Stress Apparatus 2 is operating.

Alternatively, the entire unit could be produced with a dedicated microenvironmental chamber, and the unit could be used without the cover, or without the cover and the cross brace.

In preparing the Multidirectional Shear Stress Apparatus 2 for operation, the cell laden coverslips are loaded into the wells, the Multidirectional Shear Stress Apparatus 2 is at least partially assembled, and then the medium needs to be added, but in such a way as to not introduce air, and to remove air in between the cone 16 and the dish 14. The small gap 79 between the cone radially exterior circumference 69 and the inner vertical wall 72 allows for an elegant method to both add medium and purge air bubbles. The dish 14 is set into the baseplate 8, and the cone 16 set into the dish 14 with the cross brace 100 (described below) to center the cone. At that point, the baseplate 8 is tipped up just slightly, for example, a bottle cap underneath the one side of the dish. Once tilted, culture medium is carefully pipetted in into the low end of the tilted dish 14. And then by doing so, the air underneath the cone 16 is driven out on the upper side of the dish 14 gradually. This eliminates the air that is underneath the cone 16 while filling the dish 14 with medium, because air underneath the cone 16 can cause damage to the experiment.

### Micromount Embodiment

Turning to FIG. 2, a variation of the embodiment of FIG. 1 is shown. With the Multidirectional Shear Stress Apparatus of FIG. 1, it was noted that disassembling the Multidirectional Shear Stress Apparatus to access the coverslips 86 and observe the cells was cumbersome. Thus, a variation of the Multidirectional Shear Stress Apparatus was developed. Elements of the embodiment of FIG. 2 will have the same reference numbers as the embodiment of FIG. 1, except as specifically noted.

The baseplate 8 of this embodiment is preferably sized to fit onto a stage 106 of a research microscope 108, here an inverted microscope 108. The photograph of FIG. 2 shows the gantry 12 system that was designed to hold the stepper motor 6 in place. An on-stage incubator used with this Multidirectional Shear Stress Apparatus 2 is omitted for clarity. The gantry 12 items are commercially available from Thorlabs®, and the motor 6 is purchased, but the remaining elements were the inventors' design. A magnetic coupling (not shown) was also designed by the inventors to magnetically hold the onstage incubator in place on the baseplate, but bolts, clamps, and other fasteners may be used.

The dish indentation 24 defines a through hole in the baseplate 8 sized to receive the outer vertical wall 70 of the dish 14 therewithin. At a plurality of locations around the perimeter of the dish indentation 24 are support columns 110 defining a joist passage 112 at each location for dish joists 114 and brace joists 116 to fit preferably at least partially within and provide vertical, horizontal, and rotational stability for the dish 14 and cross brace 100. Dish joists 114 extend from the outer vertical wall 70 of the dish 14. Brace joists 116 are radially terminal portions of the legs of the cross brace 100.

The wells 82 in the dish 14 of the Multidirectional Shear Stress Apparatus 2 of FIG. 2 are formed by making dish through holes 118 in the well 82 locations, and then adding a solid transparent well base 120 to each well 82. This allows the cells to be loaded on a coverslip 86, the coverslip 86 placed into a well 82 and held in place using optical coupling medium, which was found to be superior to silicon gel, the Multidirectional Shear Stress Apparatus 2 to be begin operation and medium flowing, and while the Multidirectional Shear Stress Apparatus 2 is operating, to visualize the cells responding to flow in real time, without disassembling the Multidirectional Shear Stress Apparatus 2. The transparent well bases 120 in the embodiment shown were sealed and fixed with silicone cement there was no leakage of medium from the dish 14 during operation of the Multidirectional Shear Stress Apparatus 2. The inventors were able image the responses of the same cells to both laminar or shear stress using time lapse photography since the cells are on glass coverslips 86 mounted in a coplanar manner with the upper dish surface 76, and the transparent well base 120 allowed visualization and photography from the inverted microscope 108 during operation of the Multidirectional Shear Stress Apparatus 2. The transparent well base preferably having a visible light transmittance between 0.80-0.99. The gantry 12 in the embodiment shown is actually aligned and locked in place the microscope 108 stage. The stage itself can move back and forth for scanning various wells 82 while the Multidirectional Shear Stress Apparatus 2 is operating and the cone 16 is rotating.

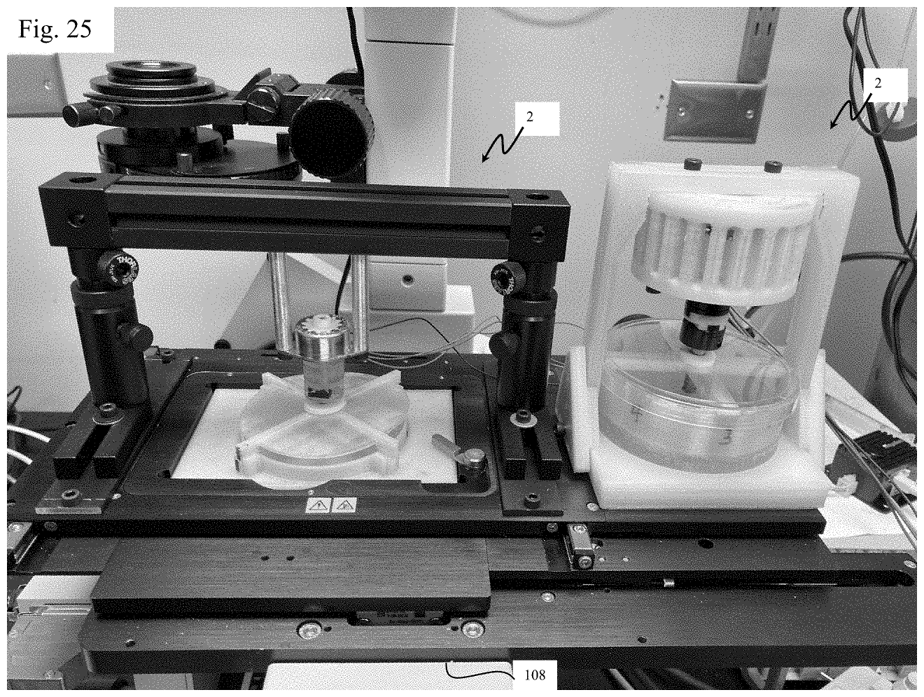

Turning to FIG. 25, a picture of both the Multidirectional Shear Stress Apparatus 2 of FIG. 1 and the Multidirectional Shear Stress Apparatus 2 of FIG. 2 are shown on the microscope 108 stage, for comparative purposes. The Multidirectional Shear Stress Apparatus 2 of FIG. 1 (right) is designed to fit within the confines of a standard cell culture incubator. The Multidirectional Shear Stress Apparatus 2 of FIG. 2 was designed to fit within the confines of a commercially available stage incubator. The on-stage incubator is not shown in the picture. The magnetic coupling is a two-part unit, both parts have magnets on their adjacent surfaces. A rational for the magnetic coupling approach was that using a magnet would allow the users to keep the cover on the onstage incubator, and the Multidirectional Shear Stress Apparatus 2 of FIG. 2 would be operating within the sealed confines of the onstage incubator and the rotary motion of the stepper motor 6 would be transferred across the lid of the incubator to the cone 16 by magnetic force. Although this approach does work, the inventors opted for a less complicated set-up for future devices if just drilling a hole through the incubator. The incubator cover will likely be made of polycarbonate, as it retains heat well, and is autoclavable.

The Multidirectional Shear Stress Apparatus 2 can be built to accommodate a wide variety of incubation systems. Some labs build an incubator around a microscope—which is expensive. The inventors used a high precision delivery system for CO₂ and heat, which gets delivered into a smaller stage (shown in FIG. 2)—the size of a small book—that is a smaller incubator designed to fit on the stage itself. To operate, the cell laden coverslips 86 are placed in the wells 82, secured in place, medium is added, air bubbles are removed, the Multidirectional Shear Stress Apparatus 2 is assembled, and then fastened to the stage. The wells 86 lineup with the microscope 108 projectors. The cells are held in place by the device and then the gas and heat are delivered to the incubator on the stage. In one experiment, cells were kept alive for two days straight while imaging them at 30 second intervals to do time-lapse photography.

The Multidirectional Shear Stress Apparatus 2 of FIG. 1 uses a commercially available Oldham® coupling to transmit rotary motion from the motor 6 to the spindle 56 of the cone 16. The coupling is direct, but does minimize any potential vibration from the motor 6. The inventors have used the Multidirectional Shear Stress Apparatus 2 of FIG. 1 in several experiments and it performs extremely well.

The stepper motor 6 used in the Multidirectional Shear Stress Apparatus of FIG. 1 is a 46000 unit from Haydon Kerk®, one of their, the step angle for the motor being 7.5 degrees. There are other manufacturers of stepper motors 8 that may be used for the Multidirectional Shear Stress Apparatus 2. The stepper motor for the Multidirectional Shear Stress Apparatus 2 of FIG. 2 is a Haydon Kerk® Z26000 series, about half the size of the 46000 series of stepper motors 8. Since it is a smaller stepper motor 6, the inventors used a smaller, lighter weight cone 16. Substantially everything but the motor 6 and shaft 18, gantry 12, cone 16, and centering pin 68 of the Multidirectional Shear Stress Apparatus of FIG. 2 was 3D printed. The cone 16 was made using a lathe, with the first angle of inclination α being approximately 4 degrees. That angle is preferably maintained in both Multidirectional Shear Stress Apparatus 2. The stepper motors 8 in the Multidirectional Shear Stress Apparatus 2 allow for micron resolution of cone 16 movement.

Turning back to FIG. 1, which is a photograph of a working embodiment of the Multidirectional Shear Stress Apparatus 2. In this embodiment, a commercially available Oldham® coupler 20 is used and a cross brace 100 was added to minimize possible cone 16 wobble due to any misalignment of the cone 16 and motor 6 along the central axis 52 of the Multidirectional Shear Stress Apparatus 2. The cross brace 100 can be made in the open format (not shown) or as shown, in a closed unit, the latter to maintain sterility of the cone/dish assembly while moving from one site to another. The stepper motor 6 is linked to a laptop computer via a USB-drive and software is used to control the function of the Multidirectional Shear Stress Apparatus 2. The Multidirectional Shear Stress Apparatus 2 is designed for prolonged exposure to conditions present in a cell culture incubator and, with the exception of the motor and its housing, all parts are preferably autoclavable and reusable. Many such autoclavable parts may be formed from polycarbonate, or another material which can be repetitively cleaned, sterilized and reused, such as the centering pin 68 and shaft 18 being made from stainless steel. Items made from polycarbonate may also be made from aluminum or stainless steel, for example.

The Multidirectional Shear Stress Apparatus 2 are shown as a direct drive, but System is direct drive, but an indirect drive system, such as gears, or preferably a belt pully system, may be used to attain finer motions with the cone 16. The belt may be flat belt, a “V” shaped belt, cog toothed belt, and helical offset tooth belt, for example, with corresponding pullies, used to transmit power from the shaft 18 to the spindle 56. This would allow for smooth and powered motion of the cone 16 with the current stepper motor 6 at speeds down to 35 rpm and 20 rpm, for example. Gears may also be used, such as epicyclic gearing to minimize space and keep the shaft 18 and spindle 56 coaxial, and helical, double helical, and herringbone gearing, to maintain the smoothness of rotation and minimize jerk of spur gear teeth engagement.

Though the Multidirectional Shear Stress Apparatus 2 are shown in a preferably relative size range, they could be made bigger (2, 3, 4 times bigger) or smaller (¾ the size, ½ the size. ¼ the size, for example), depending on the desired use. A larger size might be to accommodate larger coverslips 86 or a larger number of rings 84, 88, 90. A smaller size might be for viewing on smaller microscope 108 stages, for example.

In further embodiments, there are additional other accessory devices that could be incorporated into the Multidirectional Shear Stress Apparatus to facilitate the identification of device readouts for data collection: High resolution encoders on the motor shaft would give precise measurement/feedback to the operating system of the controller with regard to rate, direction, time of movement. Flowmeters and pressure transducers embedded in the baseplate would give measurement/feedback to the controller with regard to both flow and waveform fluidics within the culture medium under flow.

Experiments. Referring to FIGS. 24A-24F, results of laminar versus turbulent shear experiment conducted with the Multidirectional Shear Stress Apparatus 2 are shown. Panels FIG. 24A, FIG. 24C, and FIG. 24E are cultures of wild-type glomerular podocytes; panels FIG. 24B, FIG. 24D, and FIG. 24F are cultures of mutant glomerular podocytes lacking the ability to synthesize heparan sulfate. The cultures were initially grown on glass coverslips 86 and then transferred (FIG. 24C, FIG. 24D, FIG. 24E, FIG. 24F) to a Multidirectional Shear Stress Apparatus 2 of FIG. 1 for shear stress experiments, though an embodiment of the Multidirectional Shear Stress Apparatus having only six wells 82 in the dish 14. As a control, a set of each cell type (FIG. 24A, FIG. 24B) were maintained in culture under static conditions for the same time interval as the shear stress experiments were run. Since the cells were grown on coverslips 86, for each experiment, triplicate coverslips 86 of cells (wild-type or mutant) were placed in the wells 82 of the dish 14. Thus, triplicate samples for each cell type were run under the same shear conditions at the same time.

For laminar shear studies, the cone 16 was rotated in a unidirectional fashion @120 rpm for a period of 6 hours. For turbulent shear studies, the cone 16 was rotated in a bidirectional manner at the same rate; however, the extent of rotation in one direction was constrained to 6.25 rotations before a directional change was evoked, and then after 6.25 rotations in the second direction, repeated.

Although there are visible differences in the readouts of the experiment in the laminar flow group (FIG. 24C vs FIG. 24D), the most striking occurs in panels FIG. 24E and FIG. 24F, where wild-type and mutant podocytes are exposed to turbulent flow. Again, to reiterate, the wild-type and mutant podocytes were subjected to the same forces at the same time within the apparatus (Magnification 4×, Final Magnification 40×).

A purpose of Multidirectional Shear Stress Apparatus 2 is to investigate the effects of laminar or shear flow on cellular behaviors. Both of these types of flow can be found in areas of the vascular tree (arteries) Along the linear path of arteries the primary type of flow that the cells (endothelial) cells experience is laminar flow. At points in the arterial tree where the vasculature branches or bifurcates (splits into two forks) the cells in these regions experience shear/turbulent flow. It is well described in the literature that regions in the vascular tree that are subjected to shear/turbulent flow are regions that are prone to the development of atherosclerosis. Thus, this model serves as an in vitro tool to discern the effects of laminar or shear/turbulent flow on cellular functions such as gene expression, pro-inflammatory signaling responses, etc., that occur in vivo. To program turbulent flow, a processor tells motor to spin the shaft forward for a first number of rotations, optionally briefly pause, and the go backward for a second number of rotations, optionally pause, and then repeat; where the first number can be less than, the same, or greater than the second number. In a first embodiment, the first and second number are between 1.0 and 10.0, and the brief pauses are preferably between 0.2 and 1.0 seconds

Besides having relevance to investigations into the genesis of vascular diseases such as atherosclerosis, the Multidirectional Shear Stress Apparatus 2 can be used to determine the effects of variabilities (direction, rate, pulsatile force) on other cell populations that have been identified to experience flow. Such cells populations would include cells from the hepatic bile ducts, pancreatic ducts, and elements present in the nephron (podocytes, tubule epithelial cells) and cells of the urinary system (ureter, urethra). Several diseases, including polycystic kidney disease are the direct result of the inability of cells in those organs to properly sense flow conditions.

The invention illustratively disclosed herein suitably may explicitly be practiced in the absence of any element which is not specifically disclosed herein. While various embodiments of the present invention have been described in detail, it is apparent that various modifications and alterations of those embodiments will occur to and be readily apparent those skilled in the art. However, it is to be expressly understood that such modifications and alterations are within the scope and spirit of the present invention, as set forth in the appended claims. Further, the invention(s) described herein is capable of other embodiments and of being practiced or of being carried out in various other related ways. In addition, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items while only the terms “consisting of” and “consisting only of” are to be construed in the limitative sense.

### REFERENCE NUMBERS

2 Multidirectional Shear Stress Apparatus
4 Motor Mount
6 Stepper Motor
8 Baseplate
10 Support
12 Gantry
14 Dish
16 Cone
18 Shaft
20 Coupler
22 Upper Baseplate Surface
24 Dish Indentation
26 Locking Square
28 Locking Recess
30 Bottom Surface of Dish
32 First Leg
34 Second Leg
36 Crossbeam
37 Fasteners
38 Brackets
40 Interior Motor Recess
42 Motor Coupling
44 Lower Shoulder Ring
46 Upper Shoulder Ring
48 Longitudinal Slits
50 Wires
52 Central Axis
54 Upper Coupler Concavity
56 Spindle
58 Lower Coupler Concavity
60 Central Circular Cone Section
62 Lower Cone Surface
64 Oblique Cone Section
66 Upper Cone Surface
68 Centering Pin
69 Cone Radially Exterior Circumference
70 Outer Vertical Wall
72 Inner Vertical Wall
74 Rim
76 Upper Dish Surface
78 Interior of Dish
79 Gap
80 Center Notch
82 Well
84 First Ring
86 Coverslip
88 Second Ring
90 Third Ring
92 Central Circular Dish Section
94 Oblique Dish Section
96 Sensors
98 Cover
100 Cross Brace
102 Spindle Passage
104 Center Core
106 Stage
108 Microscope
110 Support Columns
112 Joist Passage
114 Dish Joists
116 Brace Joists
118 Dish Through Hole
120 Transparent Well Base

Wherefore, I/we claim:

1. A Multidirectional Shear Stress Apparatus comprising:
a baseplate;
a dish attached to the baseplate;
a motor attached to the baseplate, the motor turning a shaft;
a cone functionally and rotationally attached to the shaft; and
a first plurality of wells defined in the baseplate to receive coverslips.

2. The Multidirectional Shear Stress Apparatus of claim 1 further comprising a locking square disposed on one of a bottom surface of the dish and an upper baseplate surface, and a mating locking recess defined in the other of the bottom surface of the dish and the upper baseplate surface.

3. The Multidirectional Shear Stress Apparatus of claim 1 wherein the shaft and a spindle of the cone are coaxial along a central axis.

4. The Multidirectional Shear Stress Apparatus of claim 1 wherein a centering pin extends from a lower cone surface and mates with a center notch in the dish.

5. The Multidirectional Shear Stress Apparatus of claim 1, wherein a lower cone surface has a radially interior and substantially planar central circular cone section and a radially exterior oblique cone section.

6. The Multidirectional Shear Stress Apparatus of claim 5, wherein the oblique cone section extends linearly radially outwardly at a constant first angle of inclination from the circular cone section to a cone radially exterior circumference.

7. The Multidirectional Shear Stress Apparatus of claim 1, further comprising a gantry stationarily positioning the motor with respect to the cone.

8. The Multidirectional Shear Stress Apparatus of claim 1, further comprising a gap between an inner vertical wall of the dish and a cone radially exterior circumference, wherein the gap is between 0.50 mm and 3.00 mm.

9. The Multidirectional Shear Stress Apparatus of claim 1 further comprising a coupler connecting the shaft to a spindle of the cone.

10. The Multidirectional Shear Stress Apparatus of claim 1, wherein the first plurality of wells is equidistant from a radial center of the dish, forming a first ring.

11. The Multidirectional Shear Stress Apparatus of claim 10, further comprising a second plurality of wells equidistant from the radial center of the dish and radially spaced from the first ring.

12. The Multidirectional Shear Stress Apparatus of claim 1 wherein the motor is a stepper motor.

13. The Multidirectional Shear Stress Apparatus of claim 1 further comprising a sensor in the dish which measures one of flow speed, flow direction, flow speed and flow direction, temperature, and CO2 level.

14. The Multidirectional Shear Stress Apparatus of claim 1 further comprising an upper dish surface having a radially interior and substantially planar central circular dish section and a radially exterior oblique dish section, with the first plurality of wells being disposed in the oblique dish section.

15. The Multidirectional Shear Stress Apparatus of claim 14, wherein a lower cone surface has an oblique cone section extending linearly radially outwardly at a constant first angle of inclination from a central circular cone section, and the oblique dish section extends linearly radially outwardly at a constant second angle of inclination from the central circular dish section, and the first angle of inclination is substantially the same as the second angle of inclination.

16. The Multidirectional Shear Stress Apparatus of claim 1 further comprising a transparent well base in each of the first plurality of wells, allowing coverslips placed on the transparent well base to be viewed from below the Multidirectional Shear Stress Apparatus during operation of the Multidirectional Shear Stress Apparatus.

17. The Multidirectional Shear Stress Apparatus of claim 16 further comprising a dish indentation defining a through hole in the baseplate sized to receive and outer vertical wall of the dish therewithin.

18. The Multidirectional Shear Stress Apparatus of claim 17 further comprising support columns disposed at a plurality of locations around a perimeter of the dish indentation, the support columns defining a joist passage at each location for dish joists to fit at least partially within.

19. The Multidirectional Shear Stress Apparatus of claim 18 wherein dish joists extend from the outer vertical wall of the dish at a plurality of locations.

20. A Multidirectional Shear Stress Apparatus comprising:
a baseplate;
a dish attached to the baseplate;
a stepper motor attached to the baseplate, the motor turning a shaft;
a cone functionally and rotationally attached to the shaft;
a first plurality of wells defined in the baseplate to receive coverslips;
a dish indentation sized to receive a vertical outer wall of the dish;
one of
(a) a locking square disposed on one of a bottom surface of the dish and an upper baseplate surface, and a mating locking recess defined in the other of the bottom surface of the dish and the upper baseplate surface, and
(b) the dish indentation defining a through hole in the baseplate sized to receive and outer vertical wall of the dish therewithin and support columns disposed at a plurality of locations around a perimeter of the dish indentation, the support columns defining a joist passage at each location for dish joists to fit at least partially within;
a spindle extending from a radial center of the cone, the spindle being coaxial with the shaft along a central axis;
a coupler connecting the shaft to a spindle of the cone;
a cross brace encircling and bracing the spindle;
a gantry stationarily positioning the motor with respect to the cone;
a gap between an inner vertical wall of the dish and a cone radially exterior circumference, wherein the gap is between 0.50 mm and 3.00 mm;
a lower cone surface having a radially interior and substantially planar central circular cone section and a radially exterior oblique cone section, the oblique cone section extends linearly radially outwardly at a constant first angle of inclination from the circular cone section to a cone radially exterior circumference;
a centering pin extending from the lower cone surface and mating with a center notch in the dish;
the first plurality of wells being equidistant from a radial center of the dish and forming a first ring;
a second plurality of wells equidistant from the radial center of the dish and radially spaced from the first ring;
a sensor in the dish which measures one of flow speed, flow direction, flow speed and flow direction, temperature, and CO2 level;
a well base in each of the wells being one of opaque, translucent, and transparent, where a transparent well base allows cells on coverslips placed on the transparent well base to be viewed from below the Multidirectional Shear Stress Apparatus during operation of the Multidirectional Shear Stress Apparatus; and
a cover being coaxial with the cross brace and extending radially substantially past an internal diameter of the inner vertical wall of the dish.
